(19) United States
(12) Patent Application Publication
(10) Pub. No.: US 2026/0355418 A1
(43) Pub. Date: Nov. 12, 2026

(54) Autoinjector

(71) Applicant: BECTON DICKINSON FRANCE, Le Pont-de-Claix (FR)

(72) Inventors: Michael Fiard, Eybens (FR); Lionel Maritan, Bresson (FR)

(21) Appl. No.: 19/129,783

(22) PCT Filed: Nov. 13, 2023
(86) PCT No.: PCT/EP2023/081628

§ 371 Date: May 14, 2025

(30) Foreign Application Priority Data
Nov. 15, 2022 (EP) 22306678.8

(51) Int. Cl. A61M 5/20; A61M 5/32

(52) U.S. Cl. CPC A61M 5/2033; A61M 5/3243; A61M 2005/2073

(57) ABSTRACT

The autoinjector includes a housing extending along a longitudinal axis A and configured to receive a medical container, a needle cover coupled to and axially movable with respect to said housing between a first extended position, a retracted position, and a second extended position in which the needle cover moves back in the distal direction to shield an injection needle. The autoinjector further includes a plunger rod axially movable inside the housing between a storage position, an activation position proximally located relative to the storage position, and an injection end position distally located relative to the storage position and the activation position; a biasing member of biasing means for biasing the plunger rod in a distal direction towards the injection end position; and a locker for distally blocking the plunger rod in the storage position before activation of the autoinjector. The locker includes a first blocking member radially movable between a blocking position, in which said first blocking member prevents distal movement of the plunger rod with respect to the locker, and a release position to allow for distal movement of the plunger rod with respect to the locker. A retainer is secured to the housing for maintaining the first blocking member of the locker in the blocking position, the retainer including a lateral abutment surface for radially abutting against the locker such that the first blocking member of the locker is blocked in the blocking position, and the retainer including a proximal shoulder for axially abutting against a second blocking member of the locker. The locker is axially movable relative to the retainer together with the plunger rod between a storage position, in which the first blocking member is blocked in the blocking position, and an activation position, such that the first blocking member of the locker can move from the blocking position to the release position, movement of the locker from the storage position to the activation position being caused by the needle cover moving from the first extended position to the retracted position.

### CROSS-REFERENCE TO RELATED APPLICATIONS

This application is the United States national phase of International Patent Application No. PCT/EP2023/081628 filed Nov. 13, 2023, and claims priority to European Patent Application No. 22306678.8 filed Nov. 15, 2022, the disclosures of which are hereby incorporated by reference in their entireties.

### BACKGROUND OF THE INVENTION

Field of the Invention

The present invention relates to an autoinjector.

### Description of Related Art

In this application, the distal end of a component or of a device is to be understood as meaning the end furthest from the user's hand and the proximal end is to be understood as meaning the end closest to the user's hand. Likewise, in this application, the “distal direction” is to be understood as meaning the direction away from the user's hand, and the “proximal direction” is to be understood as meaning the direction toward the user's hand.

Automatic injection devices are designed for automatic injection of a medical product into an injection site. Autoinjectors usually comprise a housing for receiving a medical container. The medical container has a barrel defining a reservoir for containing the medical product, the barrel having a distal end provided with an injection needle and an opened proximal end receiving a plunger rod for pushing a stopper. The opened proximal end is usually provided with a flange.

Autoinjectors also include a safety shield mechanism moving from an extended to a retracted position to respectively shield or unveil the needle and an injection mechanism for automatically injecting the medical into an injection site. The injection mechanism usually includes a plunger rod for pushing a stopper inside the barrel of the medical container, and an initially compressed spring for moving the plunger in the distal direction. Locking means are provided for maintaining the plunger rod in an initial position in which the plunger rod is axially blocked despite the action of the compressed spring. A release member is typically arranged to release the plunger from the locking means and allow the spring to push the plunger rod in the distal direction to perform injection. A predetermined displacement of the safety shield towards the retracted position is required to allow the release member to unlock the locking means and release the plunger rod.

The document EP2921191 discloses a device for automatic injection of a medical product into an injection site. The device includes a two-part assembly having an upper sub-assembly assembled to a lower sub-assembly. The lower sub-assembly receives a syringe provided with an injection needle. The injection needle is covered by a rigid needle shield. The device further includes a needle cover movable relative to the housing between an extended and a retracted position. The plunger rod is coupled to a compressed spring and maintained in an initial position by a deflectable retainer of an inner cylinder. A push button is arranged at a proximal end of the device for releasing the plunger rod by deflecting the retainer outwardly. Activation of the push button only becomes possible when the needle cover has moved a predetermined distance in the proximal direction towards the retracted position. Besides, activation of the push button requires the end user to press the push button. That is, the end user must perform a specific additional operation to activate the device. Moreover, this operation may not be so easy since the end user must in the same time exert a distal force on the push button and hold the autoinjector pressed against his/her skin.

There is therefore a need for an autoinjector allowing activation of the injection mechanism without requiring an additional operation from the end user.

It is known from the document EP3384944 an autoinjector for administering a medicament.

To assess the robustness of the autoinjectors, autoinjectors are subjected to drop tests as required in ISO11608. These drop tests usually consist in dropping the autoinjectors at least once from a height of 1 m onto a horizontal floor. There are three drop directions: a drop “cap upward”, a drop “cap downward”, and a drop with the autoinjector being horizontal. The autoinjectors may be sensitive to the drop test “cap upward”.

### SUMMARY OF THE INVENTION

There is therefore a need for an autoinjector that reduces the risks of being activated during a drop test “cap upward” or any other drop.

To activate the autoinjectors, the user has to press the autoinjector against an injection site. The force exerted by the user has to overcome a predetermined activation force to get the autoinjector activated and trigger the injection. However, the activation force may be quite high to avoid activation during a drop test “cap upward”, and once overcome, there might no longer be any resistance, which makes the activation process and the consquent triggering of the injection quite sudden for the user.

There is therefore a need for a more stable activation force and a less sudden feedback for the end user.

An aspect of the invention is an autoinjector, for automatic injection of a product into an injection site, said autoinjector comprising:
a housing extending along a longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel,
a needle cover coupled to and axially movable with respect to said housing between a first extended position, in which the needle cover at least partially shields the injection needle, a retracted position, in which the needle cover moves proximally with respect to the housing, and a second extended position in which the needle cover moves back in the distal direction to shield the injection needle,
a plunger rod axially movable inside the housing between a storage position, an activation position proximally located relative to the storage position, and an injection end position distally located relative to the storage position and the activation position, the plunger rod being configured to push the stopper in order to expel the medical product when moving from the activation position to the injection end position,
a biasing member such as biasing means for biasing the plunger rod in a distal direction towards the injection end position,
a locker for distally blocking the plunger rod in the storage position before activation of the autoinjector, wherein the locker includes a first blocking member radially movable between a blocking position, in which said first blocking member axially abuts against a distal abutment surface of the plunger rod for preventing distal movement of the plunger rod with respect to the locker, and a release position, in which the first blocking member is away from the distal abutment surface of the plunger rod to allow for distal movement of the plunger rod with respect to the locker,
a retainer secured to the housing for maintaining the first blocking member of the locker in the blocking position, the retainer including a lateral abutment surface for radially abutting against the locker such that the first blocking member of the locker is blocked in the blocking position, and the retainer including a proximal shoulder for axially abutting against a second blocking member of the locker,
wherein the locker is axially movable relative to the retainer together with the plunger rod between a storage position, in which the locker radially abuts against the lateral abutment surface of the retainer, such that the first blocking member is blocked in the blocking position, and in which the second blocking member of the locker axially abuts against the proximal shoulder of the retainer such that the retainer distally blocks the locker in said storage position, and an activation position, proximally located with respect to the storage position, in which the locker is away from the lateral abutment surface of the retainer, such that the first blocking member of the locker can move from the blocking position to the release position, movement of the locker from the storage position to the activation position being caused by the needle cover moving from the first extended position to the retracted position.

The autoinjector of the invention thus allows the plunger rod to move towards the injection end position with no action from the end user other than pressing the needle cover against the injection site. Besides, the biasing means act against movement of the locker from the storage position to the activation position and therefore define the activation force. As a result, the activation force is more stable and this prevents inadvertent activation of the autoinjector during drop tests or any accidental drop. The end user can further feel the resistance of the biasing means when applying the needle cover against the injection site, and this resistance lasts from the storage position to the activation position of the locker, which provides the end user with a continuous tactile feedback that the activation process is being engaged, until activation of the autoinjector, i.e. release of the biaising means and the plunger rod which starts the injection process.

The autoinjector of the invention may further include some or all of the features below.

In an embodiment, the first blocking member defines a proximal abutment surface for axially abutting against the distal abutment surface of the plunger rod, said proximal abutment surface of the first blocking member and/or said distal abutment surface of the plunger being inclined. The first blocking member can move to the release position under the pressure transmitted by the biasing means to the plunger rod. Preferably, the distal abutment surface of the plunger rod and the abutment surface of the first blocking member are complementarily shaped, for instance a frustoconical shape. The locker may include two diametrically opposite first blocking members and/or two diametrically opposite second blocking members. The retainer may include two diametrically opposite lateral abutment surfaces. The inner diameter defined between the two lateral abutment surfaces is lower than the diameter of the portion of larger diameter of the retainer.

In an embodiment the first blocking member is arranged on an inner side of an axially extending flexible leg of the locker, and an outer side of the flexible leg defines an outer abutment surface configured for abutting against the lateral abutment surface of the retainer when the locker is in the storage position. The outer abutment surface may be defined by a radially outward protrusion. The radially outward protrusion may define a guiding proximal surface, which may be inclined with regard to the longitudinal axis A for easing insertion of the locker in the proximal direction inside the retainer. The flexible leg may be resiliently deformable. The flexible leg has a fixed proximal end and a free distal end.

In an embodiment, the first blocking member is located at a distal end of the flexible leg, and the flexible leg is delimited between two axial slots. The axial slot has a closed proximal end and an open distal end. This eases deformation of the flexible leg between the blocking position and the release position. The locker includes two diametrically opposite flexible legs and two pairs of axial slots delimiting said flexible legs. The first blocking member may be a radially inward protrusion.

Possibly, the release position of the first blocking member may correspond to a deformed position of the flexible leg, while the blocking position of the first blocking member may correspond to a rest position of the flexible leg. Alternatively, the release position of the first blocking member may correspond to a rest position of the flexible leg, while the blocking position of the first blocking member may correspond to a deformed position of the flexible leg.

In an embodiment, the second blocking member is located on an outer surface of the flexible leg of the locker. That is, on a distal portion of the locker. The second blocking member may be in the form of a circumferentially extending rib. The axial distance between the second blocking member and a distal end of the flexible leg is lower than the axial distance between the storage position and the activation position of the locker. The second blocking member includes a distal stop configured for abutting against the proximal shoulder of the retainer when the locker is in the storage position. The distal stop may be orthogonal to the longitudinal axis A.

In an embodiment, the locker includes a driving leg for receiving a proximal push of the needle cover when the needle cover moves from the first extended position to the retracted position. The driving leg may be separated from the flexible leg by one of said two axial slots. The driving leg defines a distal abutment surface configured for abutting against the needle cover when the needle cover moves from the first extended position to the retracted position. The distal abutment surface may be the distal-most surface of the locker. The locker includes two diametrically opposite driving legs. Preferably, the driving leg(s) and the flexible leg(s) of the locker are circumferentially distributed 90° from each other. The driving legs may be distal to the flexible legs. Possibly, the locker includes an outwardly extending radial arm connecting the driving leg to an outer surface of a lateral wall of the locker. The outwardly extending radial arm may define a proximal stop that may abut against the retainer when the locker is in the activation position.

In an embodiment, the locker has a cylindrical guiding portion and the retainer has one or more cylindrical centering surfaces configured for guiding axial movement of the locker inside the retainer between the storage position and the activation position. The retainer may include two diametrically opposite centering surfaces. Possibly, the guiding portion of the locker is configured such that the locker can also rotate inside the retainer with respect to the longitudinal axis A. For instance, the guiding portion is devoid of any protrusion. The centering surfaces are proximal to the portion of larger diameter of the retainer.

In an embodiment, the guiding portion of the locker includes includes anti-rotation means for blocking rotation of the locker relative to the retainer.

Possibly, the retainer is in the form of a tubular sleeve having an opened distal end and closed proximal end secured to a transversal wall of the housing. The retainer may further be connected to a lateral wall of the housing by one or more stiffeners.

In an embodiment, the proximal shoulder is arranged proximal to the one or more centering surfaces. The proximal shoulder is arranged at a proximal portion, i.e. a proximal half, of the retainer. In this case, the proximal side of the radially inward protrusion may be inclined with respect to the longitudinal axis A.

Possibly, the retainer includes an axial cutout for receiving the driving leg of the locker when the locker is in the activation position. The axial cutout may have an opened distal end and a closed proximal end that is delimited by a distal stop which may be configured for abutting against the proximal stop of the driving leg.

In an embodiment, the lateral abutment surface of the retainer is defined by a radially inward protrusion, and proximal to said radially inward protrusion, the retainer has a portion of larger diameter configured for receiving the first blocking member when the first blocking member of the locker moves to the release position. That is, the portion of larger diameter provides room for the outward deformation of the locker. The radially inward protrusion may be arranged at a distal end of the retainer. The portion of larger diameter of the retainer has a greater diameter than a diameter defined between the lateral abutment surfaces. The portion of larger diameter is arranged proximal to the lateral abutment surfaces.

In an embodiment, the proximal shoulder of the retainer is located on a proximal side of the radially inward protrusion of the retainer. The proximal shoulder may be thus distal to the centering surfaces.

In an embodiment, the second blocking member is a resiliently deformable tab. The second blocking member may be away from the flexible leg of the locker. The second blocking member may be circumferentially offset the first blocking member, i.e. not axially aligned with the first blocking member, but may instead be aligned with the driving leg. The second blocking member may be located in a proximal portion, i.e. in a proximal half, of the locker. The second blocking member includes a distal stop configured for abutting against the proximal shoulder of the retainer when the locker is in the storage position. The distal stop may be orthogonal to the longitudinal axis A.

In an embodiment, the housing has a containment wall configured to laterally retain a proximal end of the biasing means. The containment wall may be in the form of an annular rib that distally protrudes from a transversal wall forming a distal end of the housing. Preferably, the containment wall and the transversal wall of the housing are made of a single piece.

In an embodiment, the retainer and the housing are made of a single piece. Alternatively, the retainer and the housing are two distinct parts and a proximal end of the retainer is secured to the housing by any appropriate means such as, for instance, clipping means, friction-fit, or gluing. In an embodiment, the transversal wall and a lateral wall of the housing are made of a single piece. In another embodiment, the transversal wall and the lateral wall of the housing are made of two distinct components secured to each other by any appropriate means such as, for instance, clipping means, friction-fit, or gluing. The transversal wall such forms a cap for closing the proximal end of the housing.

Possibly, the plunger rod extends within the biasing means. Alternatively, the biasing means extend inside the plunger rod.

In an embodiment, movement of the locker from the storage position to the activation position is opposed by the biasing means only. There are no dots or protrusions that would hinder movement of the locker from the storage position to the activation position.

### BRIEF DESCRIPTION OF THE DRAWINGS

The terms Fig., Figs., Figure, and Figures are used interchangeably in the specification to refer to the corresponding figures in the drawings.

The invention and the advantages arising therefrom will clearly emerge from the detailed description that is given below with reference to the appended drawings as follows:

FIG. 1A is a perspective view of an autoinjector according to an embodiment of the invention,

FIG. 1B is a top view of an autoinjector according to an embodiment of the invention,

FIG. 2 is an exploded view of an autoinjector according to an embodiment of the invention,

FIG. 3 is a perspective view of a top body of an autoinjector according to the embodiment of FIG. 2,

FIGS. 4A and 4B are perspective views of a locker of an autoinjector according to the embodiment of FIG. 2,

FIGS. 5A and 5B are cross section views of the autoinjector of FIG. 2, respectively along the lines A-A and B-B of FIG. 1B, the locker and the plunger rod being in the storage position,

FIGS. 6A and 6B are cross section views of the autoinjector of FIG. 2, respectively along the lines A-A and B-B of FIG. 1B, the locker and the plunger rod being in the activated position,

FIG. 7 is a perspective view of a top body of an autoinjector according to another embodiment of the invention,

FIGS. 8A and 8B are perspective views of a locker of an autoinjector according to the embodiment of FIG. 7,

FIGS. 9A and 9B are cross section views of the autoinjector according to the embodiment of FIG. 7, respectively along the lines A-A and B-B of FIG. 1B, the locker and the plunger rod being in the storage position,

FIGS. 10A and 10B are cross section views of the autoinjector according to the embodiment of FIG. 7, respectively along the lines A-A and B-B of FIG. 1B, the locker and the plunger rod being in the activated position,

FIGS. 11A and 11B are, respectively, a cross-section view and a perspective view of a plunger rod of an autoinjector according to an embodiment of the invention.

### DESCRIPTION OF THE INVENTION

The different features of the embodiments can be used in combination with and used with other embodiments as long as the combined parts are not inconsistent with or interfere with the operation of the device and assembly. The phraseology and terminology used herein is for the purpose of description and should not be regarded as limiting. The use of “including,” “comprising,” or “having” and variations thereof herein is to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not limited to physical or mechanical connections or couplings. As used herein, the singular forms “a,” “an,” and “the” are intended to include the plural forms as well, unless the context clearly indicates otherwise. As used herein, the term “and/or” includes any and all combinations of one or more of the associated listed items. It will be understood that, although the terms first, second, etc., may be used herein to describe various elements, these elements should not be limited by these terms. These terms are only used to distinguish one element from another. For example, a first element could be termed a second element, and, similarly, a second element could be termed a first element without departing from the scope of the present disclosure.

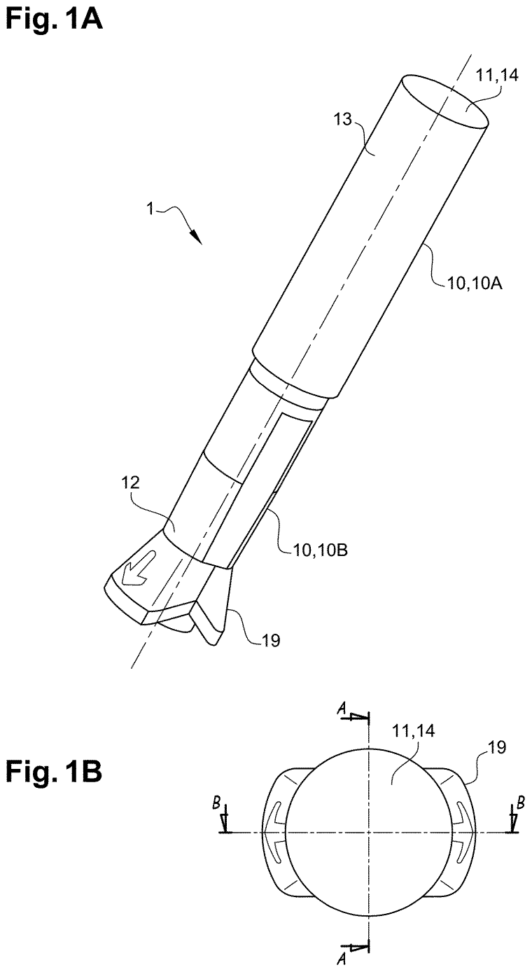

With reference to FIGS. 1A, 1B is shown an autoinjector 1 according to an embodiment of the invention. The autoinjector 1 is designed for automatic injection of a product into an injection site. The autoinjector 1 extends along a longitudinal axis A. The autoinjector 1 includes a housing 10 comprising a top body 10A and a bottom body 10B assembled to each other by any appropriate securing means such as, for instance, snap-fitting means. The housing 10 has a proximal end 11 and an opposite distal end 12. A cap 19 is removably attached to the distal end 12. The top body 10A has a cylindrical lateral wall 13 and a transversal wall 14 that forms the closed and fixed proximal end 11 of the housing 10. As illustrated herein, the transversal wall 14 and the lateral wall 13 of the housing 10 are made of a single piece. In an alternative embodiment (not shown), the transversal wall 14 and the lateral wall 13 of the housing 10 may be made of two distinct parts secured to each other by any appropriate means such as, for instance, snap-fitting means, friction-fit, or gluing.

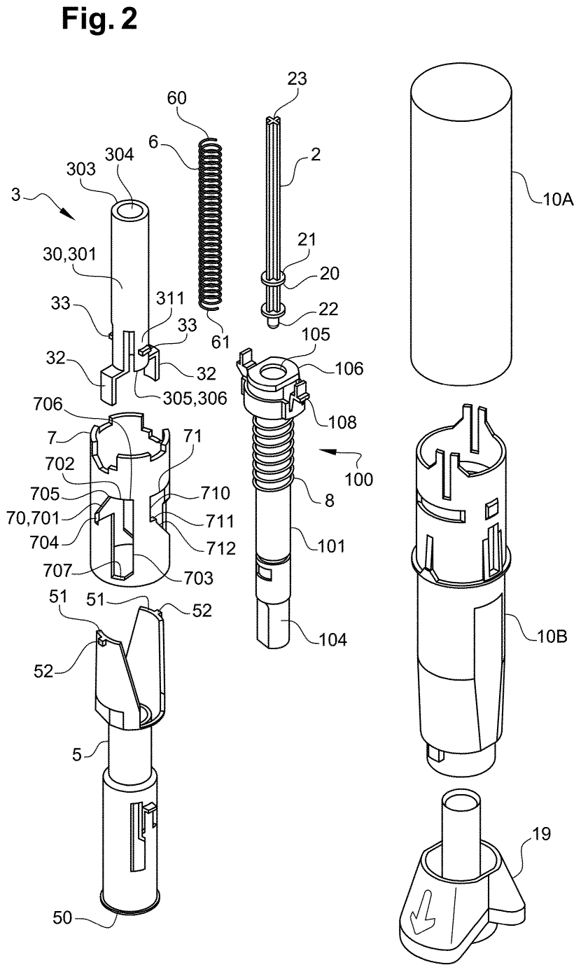

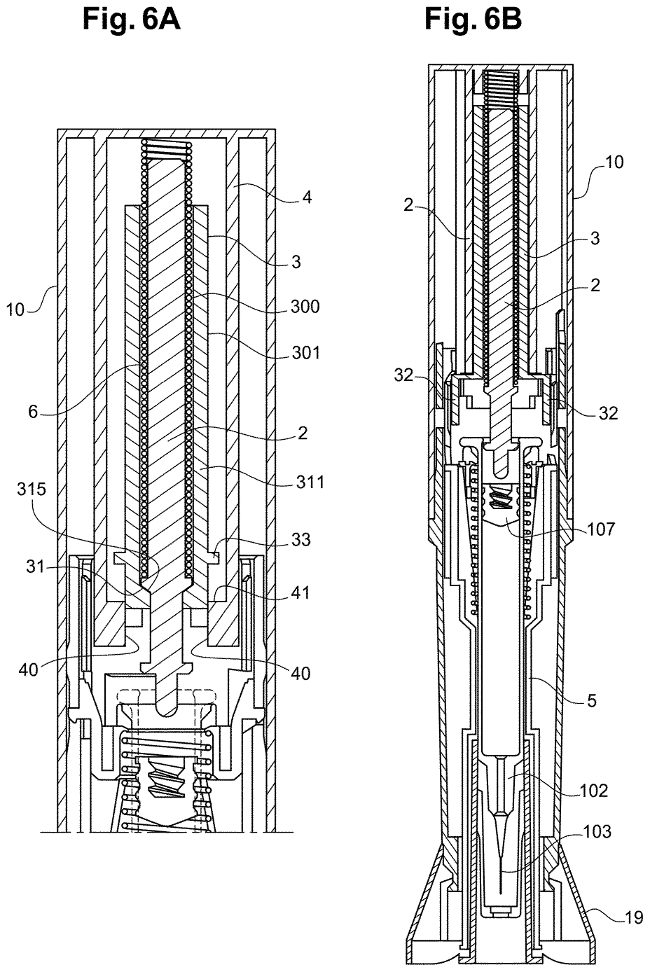

FIG. 2 illustrates an exploded view of the autoinjector 1. The bottom body 10B is configured to receive a medical container 100, such as a syringe. For instance, the medical container 100 may be a prefilled syringe. The medical container 100 has a tubular barrel 101 defining a reservoir for containing a medical product to be injected. The barrel 101 has a distal end 102 provided with an injection needle 103, FIG. 6B, and a needle shield 104 removably attached to said distal end 102 for protecting and sealing the injection needle 103. The barrel 101 has an opened proximal end 105 which may be provided with a flange 106 and which is configured to receive a plunger rod 2 urged in the distal direction by a biasing member or biasing means which may be in the form of an injection spring 6. The injection spring 6 has a distal end 61 abutting against (a radial flange 21 of) the plunger rod 2 and a proximal end 60 abutting against the housing 10 (and more specifically against the transversal wall 14). A ring 108 may be secured to the proximal end 105 of the barrel 101.

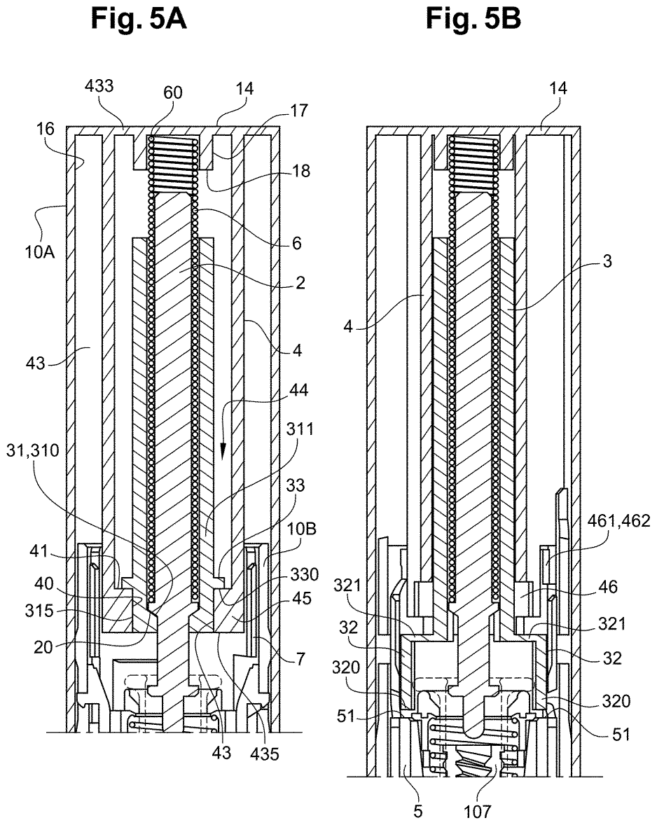

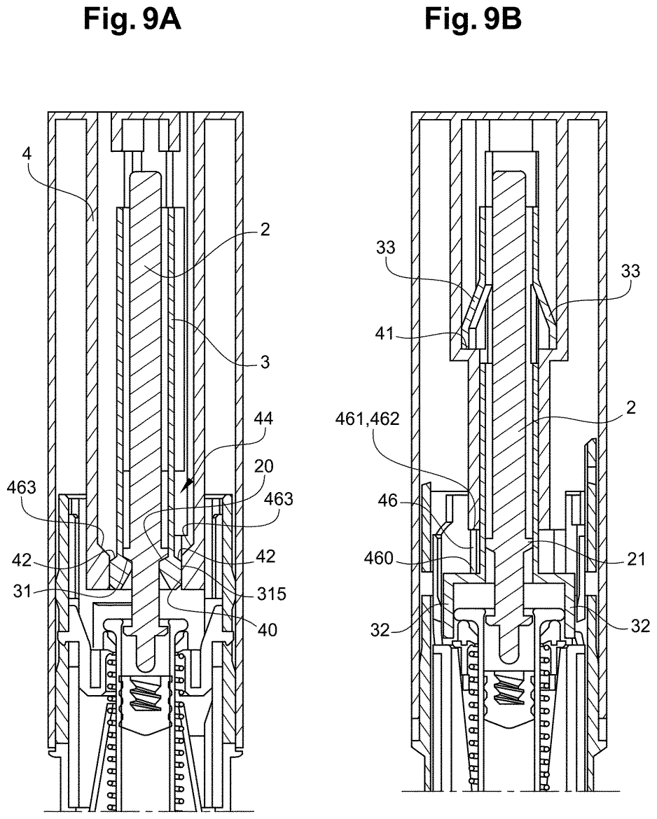

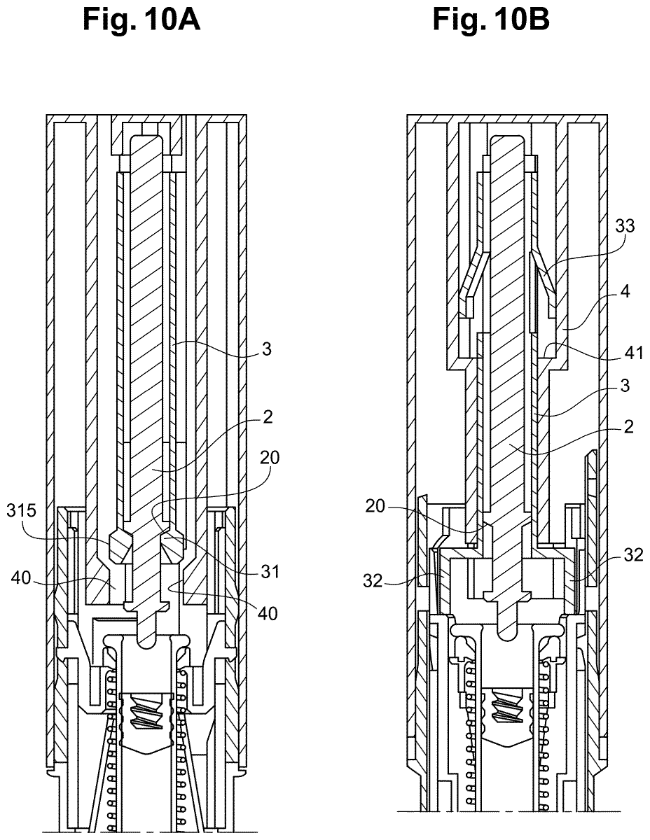

The plunger rod 2 is configured to push a stopper 107, FIG. 6B, arranged inside the barrel 101, thereby expelling the medical product. The plunger rod 2 includes a distal abutment surface 20 whose function will be described below. The plunger rod 2 is axially movable inside the housing 10 between a storage position (FIGS. 5A-5B, 9A-9B), an activation position (FIGS. 6A-6B, 10A-10B) proximally located relative to the storage position, and an injection end position (not shown) distally located relative to the storage position and the activation position. In the injection end position, the plunger rod 2 may press the stopper 107 against the distal end of the reservoir formed by the barrel 101.

The autorinjector further includes a needle cover 5 arranged within the bottom body 10B. The needle cover 5 has a distal end 50 designed to be pressed against an injection site, such as a user's skin, a radial tab 52 for engaging a first slot 70 of a cam 7, and a proximal pushing surface 51 for abutting against a locker 3, as will explained in further details below. The contact between the needle cover 5 and the locker 3 may be direct, i.e. without any intervening parts. The proximal pushing surface 51 may extend in a circumferential direction, apart from the top surface of the tabs 52 which may define a radial portion of the pushing surface 51 for ensuring abutment between the needle cover 5 and the locker 3. The needle cover 5 is axially movable along the longitudinal axis A with respect to the housing 10 between a first extended position (pre-use position, FIGS. 5A-5B, 9A-9B), in which the needle cover 5 at least partially or completely shields the injection needle 103, a retracted position (FIGS. 6A-6B, 10A-10B), proximally located relative to said first extended position, in which the needle cover 5 activates the autoinjector 1 and thus triggers the injection, and a second extended position (safety position, not shown) in which the needle cover 5 moves back in the distal direction to safely shield the injection needle 103. Movement of the needle cover 5 from the first extended position to the retracted position is caused by the user pressing the distal end 50 of the needle cover 5 against the injection site, and movement of the needle cover 5 from the retracted position to the safety position is due to a safety spring 8 pushing the needle cover 5 back in the distal direction when the user removes the injection needle 103 from the injection site.

As illustrated in FIG. 2, the autoinjector 1 may include a cam 7. The cam 7 is in the form of a cylindrical ring provided with a first slot 70 and a second slot 71. The first slot 70 has an oblique portion 701 extending between a first end 704 and a second end 705, a transversal portion 702 extending from the second end 705 to a third end 706, and an axial portion 703 extending from the third end 706 to a fourth end 707. The first slot 70 is engaged by the radial tab 52 of the needle cover 5, said radial tab 52 being located at the first end 704 when the needle cover 5 is in the first extended position, at the second end 705 and at the third end 706 when the needle cover 5 is in the retracted position, and close to or at the fourth end 707 when the needle cover 5 is in the safety position. The second slot 71 includes an axial portion 710 including a proximal shoulder 711, and an oblique portion 712 extending from said proximal shoulder 711. The second slot 71 is engaged by the ring 108, which extends in the axial portion 710 when the needle cover 5 is in the first extended position. The cam 7 is pivotally mounted inside the housing 10. Rotation of the cam 7 is first caused by the radial tab 52 sliding against the oblique portion 701 when the needle cover 5 moves from the first extended position to the retracted position, then by the ring 108 sliding against the oblique portion 712 when the ring 108 is pushed distally together with the medical container 100 by the injection spring 6. This second rotation of the cam 7 causes the radial tab 52 to move from the second end 705 to the third end 706, so that the radial tab 52 is then allowed to slide in the axial portion 703 when the needle cover 5 moves from the retracted position to the safety position.

With reference to FIG. 2 and FIGS. 4A-4B, the autoinjector 1 includes a locker 3. The locker 3 is arranged inside the top body 10A for distally blocking the plunger rod 2 in the storage position before activation of the autoinjector 1. The locker 3 may be in the form of a tubular sleeve. The locker 3 may have a cylindrical lateral wall 30, having an inner surface 300 and an outer surface 301. The inner surface 300 defines an inner cavity 302 for receiving the plunger rod 2 and the biasing means. The inner cavity 302 has a proximal end 303, that may define a proximal opening 304 allowing for insertion of the plunger rod 2 and insertion of the biasing means inside the locker 3, and an opposite distal end 305, that may define a distal opening 306 allowing for extension of the plunger rod 2 outside the locker 3 when the plunger rod 2 moves from the activation position to the injection end position. The first blocking member 31 may extend at the distal end 305 of the inner cavity 302.

The locker 3 is arranged inside the retainer 4 and axially movable together with the plunger rod 2 with respect to the retainer 4 between a storage position, FIGS. 5A-5B, in which the locker 3 prevents the plunger rod 2 from moving to the injection end position, and an activation position, FIGS. 6A-6B, proximally located with respect to the storage position, in which the locker 3 no longer prevents the plunger rod 2 from moving to the injection end position. In the activation position of the locker 3 and of the plunger rod 2, the biasing means may be totally compressed.

The locker 3 includes a first blocking member 31 which is radially deformable between a blocking position, in which said first blocking member 31 axially abuts against the distal abutment surface 20 of the plunger rod 2 for preventing distal movement of the plunger rod 2, and a release position, in which the first blocking member 31 is away from the distal abutment surface 20 of the plunger rod 2 to allow for distal movement towards the injection end position. The top body 10A includes a retainer 4, FIG. 5A, including a lateral abutment surface 40 for maintaining the first blocking member 31 of the locker 3 in the blocking position.

With reference to FIGS. 4A-4B, the first blocking member 31 is in the form of a radially inward protrusion, protruding from an inner surface 300 of the locker 3 and more specifically from an inner surface of a flexible leg 311 of the locker 3. The first blocking member 31 defines a proximal abutment surface 310 for axially abutting against the distal abutment surface 20 of the plunger rod 2, thereby axially blocking the plunger rod 2 in the storage position before activation of the autoinjector 1. The proximal abutment surface 310 may be inclined. The distal abutment surface 20 of the plunger rod 2 may also be inclined; preferably, the distal abutment surface 20 of the plunger rod 2 and the abutment surface of the first blocking member 31 are complementarily shaped, for instance a frustoconical shape. This favors movement of the first blocking member 31 from the blocking position to the release position.

The locker 3 may include two pairs of axial slots 312, each pair of axial slots 312 defining a flexible leg 311 from which protrudes the first blocking member 31. The first blocking member 31 may be located at a distal end 316 of the flexible leg 311. The axial slots 312 have a closed proximal end 313 and an open distal end 314. This eases deformation of the flexible leg 311 between the blocking position and the release position. The flexible legs 311 have a fixed proximal end 317.

It is contemplated that the release position of the first blocking member 31 may correspond to a deformed position of the flexible leg 311, while the blocking position of the first blocking member 31 may correspond to a rest position of the flexible leg 311. Therefore, movement of the first blocking member 31 from the blocking position to the release position is due to the distal pressure of the biaising means pushing the distal abutment surface 20 of the plunger rod 2 against the first blocking member 31. In another embodiment, the release position of the first blocking member 31 may correspond to a rest position of the flexible leg 311, while the blocking position of the first blocking member 31 may correspond to a deformed position of the flexible leg 311. In this case, the movement of the first blocking member 31 from the blocking position to the release position may be due to the resilient legs automatically returning to their natural rest position.

Still with reference to FIGS. 4A-4B, the locker 3 may include driving legs 32 for abutting against the proximal pushing surface 51 of the needle cover 5 when the needle cover 5 moves from the first extended position to the retracted position. The driving legs 32 accordingly include a distal abutment surface 320 that receives the proximal push of the needle cover 5. The distal abutment surface 320 may be the distal-most surface of the locker 3. The driving legs 32 may be radially offset with respect to the flexible legs 311. The locker 3 may thus include an outwardly extending radial arm 321 connecting the driving leg 32 to a lateral wall of the locker 3. The outwardly extending radial arm 321 may define a proximal stop 322 that may abut against the retainer 4 when the locker 3 is in the activation position. In the storage position of the locker 3 and in the first extended position of the needle cover 5, an axial gap may exist between the proximal pushing surface 51 of the needle cover 5 and the driving legs 32 of the locker 3.

With reference to FIG. 5A, the locker 3 has, radially opposite the first blocking member 31, an outer abutment surface 315 configured for abutting against a lateral abutment surface 40 of the retainer 4 when the locker 3 is in the storage position. The abutment between this outer abutment surface 315 of the flexible leg 311 and the lateral abutment surface 40 of the retainer 4 maintains the first blocking member 31 in the blocking position. With reference to FIG. 9A, the outer abutment surface 315 may be defined by a radially outward protrusion. The radially outward protrusion may define a guiding proximal surface 42, which may be inclined with regard to the longitudinal axis A for easing insertion of the locker 3 in the proximal direction inside the retainer 4.

A distal end 316 of the flexible legs 311 may include a chamfer 318 to ease passage of the first blocking member 31 from the blocking position to the release position.

With reference to FIGS. 4A-4B, 5A-5B, and 6A-6B, the locker 3 includes a second blocking member 33 configured for distally blocking the locker 3 in the storage position. The second blocking member 33 may be located on an outer surface of the flexible leg 311 and may be in the form of a circumferentially extending rib. The axial distance between the second blocking member 33 and a distal end 316 of the flexible leg 311 is lower than the axial distance between the storage position and the activation position of the locker 3. The second blocking member 33 includes a distal stop 330 configured for abutting against a proximal shoulder 41, FIG. 5A, of the retainer 4 when the locker 3 is in the storage position.

As illustrated in FIGS. 4A-4B, the outer surface of the lateral wall of the locker 3 may define a cylindrical outer surface forming a guiding portion 307 configured for guiding the axial movement of the locker 3 inside the retainer 4 between the storage position and the activation position. The guiding portion 307 is configured to slide against the retainer 4. In the embodiment of FIGS. 4A-4B, the guiding portion 307 is configured such that the locker 3 can also rotate inside the retainer 4 with respect to the longitudinal axis A. For instance, the guiding portion 307 is devoid of any protrusion. This permits assembly of the locker 3 inside the retainer 4 by translation, and then rotation.

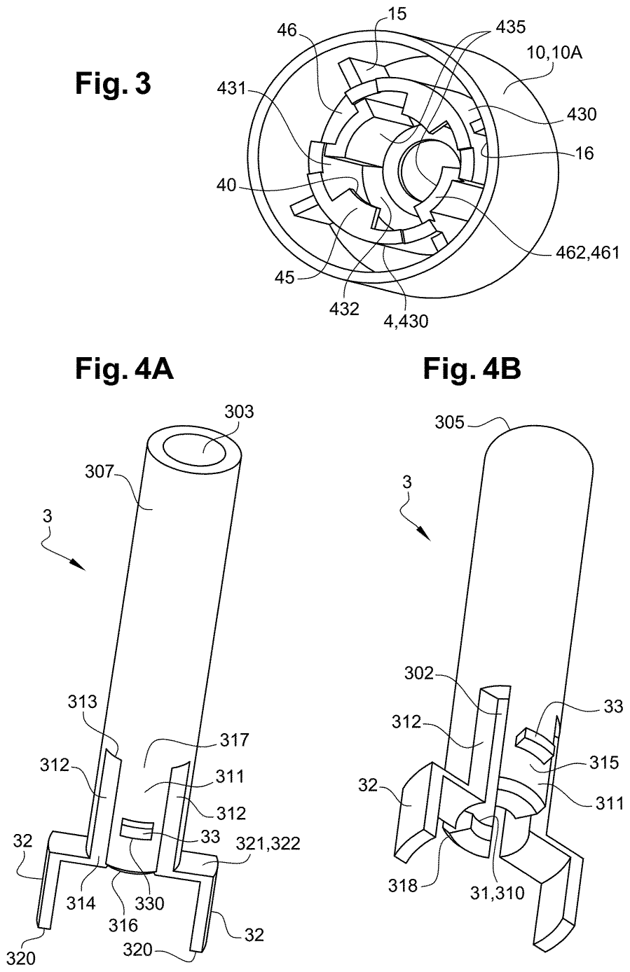

FIGS. 3, 5A-5B and 6A-6B illustrate the retainer 4. The retainer 4 may be in the form of a tubular sleeve axially extending inside the housing 10. The retainer 4 has a cylindrical lateral wall 43 defining an outer surface 430 and an inner surface 431. The outer surface 430 may be connected to the lateral wall 13 of the housing 10 by one or more stiffeners 15. The stiffeners 15 may be axially extending walls which run alongside an inner surface 16 of the housing 10 and the outer surface of the retainer 4. The stiffeners 15 may be regularly distributed around the longitudinal axis A and may extend in a virtual longitudinal plane including the longitudinal axis A. For instance, the autoinjector 1 has four stiffeners 15, but their number and shape may vary.

The inner surface 431 of the retainer 4 defines an inner cavity 432 having a closed proximal end 433 and an open distal end 434. The proximal end 433 may be closed by the transversal wall 14 of the housing 10. The opened distal end 434 may be configured to allow for axial insertion of the locker 3, and/or the plunger rod 2 and biasing means, inside the retainer 4. The opened distal end 433 is also configured to allow for extension of the plunger rod 2 outside the retainer 4 when the autoinjector 1 is activated, i.e. when the plunger rod 2 moves from the activation position to the injection end position under the distal pressure exerted by the biasing means.

The inner cavity of the retainer 4 includes two cylindrical centering surfaces 435 configured for guiding the axial movement, and if need be the rotation, of the locker 3 inside the retainer 4. The cylindrical centering surfaces 435 accordingly engage the mounting portion of the locker 3; they may have complementary shapes. In the illustrated embodiments, the retainer 4 includes two diametrically opposite centering surfaces 435. The diameter between the two centering surfaces 435 may be lower than a diameter in the rest of the inner cavity of the retainer 4; the highest of said diameters is configured to allow movement of the locker 3 from the storage position to the release position inside the retainer 4. As visible in FIGS. 5A, 6A, the inner cavity of the retainer 4 provides room for allowing outward deformation of the flexible legs 311 of the locker 3 from the blocking position to the release position.

With reference to FIGS. 5A, 6A, the retainer 4 includes a lateral abutment surface 40 for radially abutting against the first blocking member 31, more specifically against the outer abutment surface 315 of the locker 3, such that the first blocking member 31 is blocked in the blocking position as long as the locker 3 is in the storage position. The lateral abutment surface 40 may be defined by a radially inward protrusion 45 that protrudes from the inner surface 431 of the retainer 4. The radially inward protrusion 45 may be arranged at a distal end 434 of the retainer 4. When the locker 3 is moved to the activation position, the lateral abutment surface 40 of the retainer 4 no longer abuts against the first blocking member 31, so that the first blocking member 31 of the locker 3 can outwardly deflect to the release position.

The retainer 4 further includes a proximal shoulder 41 for axially abutting against a second blocking member 33 of the locker 3. The proximal shoulder 41 may be defined by a proximal side of the radially inward protrusion 45. In the storage position, the second blocking member 33 of the locker 3 axially abuts against said proximal shoulder 41. In the activation position, the second blocking member 33 of the locker 3 may be axially away from the proximal shoulder 41.

In the embodiment of FIGS. 3 to 6B, assembly of the locker 3 inside the retainer 4 is done by axially inserting the locker 3 inside the retainer 4, the second blocking member 33 of the locker 3 being circumferentially offset the radially inward protrusion 45 of the retainer 4 so that the second blocking member 33 may proximally pass beyond the radially inward protrusion 45, and then rotate the locker 3 inside the retainer 4 so that the second blocking member 33 axially faces the radially inward protrusion 45. As a result, the second blocking member 33 can abut against the proximal shoulder 41 of the retainer 4 to block the locker 3 in the storage position.

As illustrated in FIG. 3, the retainer 4 may include one or more axial cutouts 46 for receiving the driving legs 32 of the locker 3. The axial cutouts 46 may have an opened distal end 460 and a closed proximal end 461 that delimits a distal stop 462 which may be configured for abutting against the proximal stop of the driving leg 32 when the locker 3 is moved in the proximal direction. Anyway, the axial gap between the proximal stop of the driving legs 32 of the locker 3 in the storage position and the distal stop 462 of the cutouts 46 is equal to or higher than the axial distance that the locker 3 has to travel from the storage position to activation position.

In the illustrated embodiments, the retainer 4 and the housing 10 are made of a single piece. Alternatively, the retainer 4 and the housing 10 may be two distinct parts and a proximal end 433 of the retainer 4 may be secured to the housing 10 by any appropriate means such as, for instance, snap-fitting means, friction-fit, or gluing.

As visible in FIG. 3, the housing 10 may have a containment wall 17 configured to laterally retain a proximal end 60 of the biasing means. The containment wall 17 may be in the form of an annular rib that distally protrudes from the transversal wall 14 of the housing 10. Preferably, the containment wall 17 and the transversal wall 14 of the housing 10 are made of a single piece. The containment wall 17 may define a distal stop 18 for abutting against the locker 3 when the locker 3 is moved in the proximal direction. Anyway, the axial gap between the proximal end 303 of the locker 3 in the storage position and the containment wall 17, or the transversal wall 14, is equal to or higher than the axial distance that the locker 3 has to travel from the storage position to activation position.

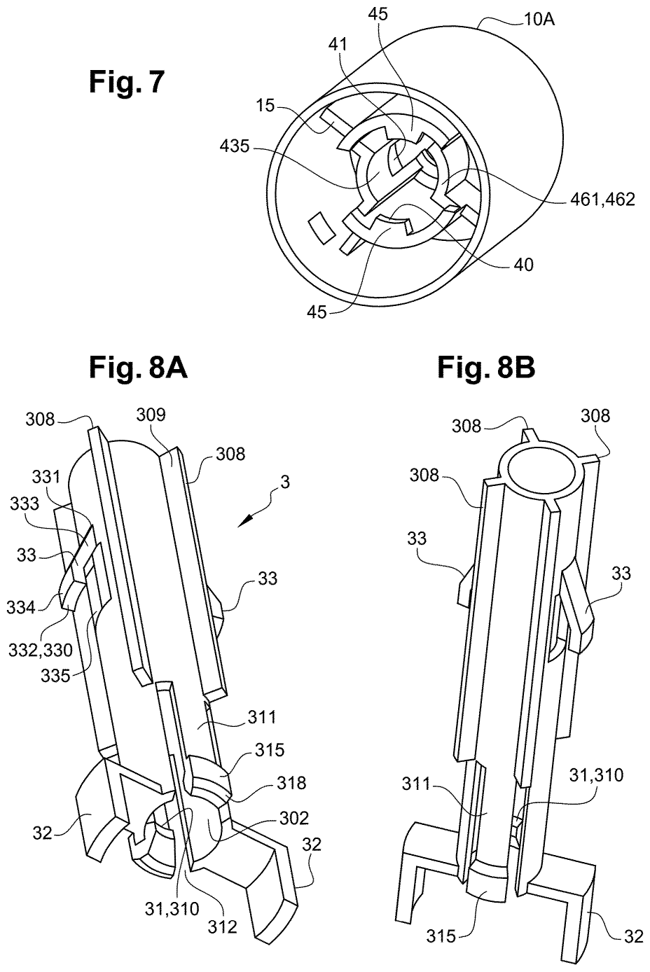

FIGS. 7, 8A-8B, 9A-9B, 10A-10B illustrate another embodiment. As best shown in FIG. 8A or 8B, the second blocking member 33 of the locker 3 may here be in the form of a an axially extending tab, instead of a circumferential rib. Besides, the tab may be located in a proximal portion, i.e. in a proximal half, of the locker 3. The second blocking member 33 (the tab) includes a distal stop 330 configured for abutting against the proximal shoulder 41 of the retainer 4 when the locker 3 is in the storage position (FIGS. 5A-5B, 9A-9B). The tab may include a first end 331 fixed to the outer surface 301 of the locker 3, and a second end 332 which may be free and which defines the distal stop 330. The first end 331 may be proximal while the second end 332 is distal. The tab may include an oblique portion 333, which may extend from the fist end 331 and inclined with regard to the longitudinal axis A, and an axial portion 334, which may extend from the oblique portion 333 to the second end 332 parallel to the longitudinal axis A. The second blocking member 33 (the tab) may be resiliently deformable between a retracted position (not shown), in which the tab is flexed radially inwardly to permit insertion in the proximal direction of the locker 3 inside the retainer 4, and an extended position (FIGS. 8A-8B, 9B, 10B), in which the tab extends radially outwardly from the retracted position, such that the distal stop 330 of the tab axially faces the proximal shoulder 41 of the retainer 4 and can abut against said proximal shoulder 41 when the locker 3 is in the storage position. The lateral wall of the locker 3 may include a cutout 335 that provides room for deformation of the second blocking member 33 in the retracted position.

Still with reference to FIGS. 8A-8B, the guiding portion 307 of the locker 3 may include anti-rotation means for blocking rotation of the locker 3 relative to the retainer 4. Thus, the first blocking member 31 cannot, by rotation, be shifted away from the of the retainer 4. Preferably, the anti-rotation means may be configured to rotationnally position the locker 3 with respect to the retainer 4 before assembly, that is to align the first blocking member 31 of the locker 3 with the lateral abutment surface 40 of the retainer 4 and the second blocking member 33 of the locker 3 with the proximal shoulder 41 of the retainer 4. The anti-rotation means may be in the form of axially extending ribs 308 which protrude from the outer side of the lateral wall of the locker 3. The axially extending ribs 308 define orthoradial stop 309s that abut against the retainer 4, more specifically against corresponding abutment surfaces at both sides of the centering surfaces 435, for blocking rotation of the locker 3 relative to the retainer 4.

As illustrated in FIGS. 7, 9B, 10B, the proximal shoulder 41 of the retainer 4 is arranged at a proximal portion, i.e. a proximal half, of the retainer 4. In this case, a proximal side of the radially inward protrusion 45 of the retainer 4 may define a ramped surface 463 which is inclined with respect to the longitudinal axis A.

Assembly of the locker 3 inside the retainer 4 is here done by translation only of the locker 3 inside the retainer 4. The second blocking members 33 are forced in the retracted position by abutment of the oblique portion against the distal stop 462 and then slide against the centering surfaces 435 of the retainer 4 until they reach a portion 44 of larger diameter of the inner cavity, said diameter being higher than the diameter between the centering surfaces 435 of the retainer 4. The portion 44 of larger diameter of the inner cavity is separated from the centering surfaces 435 by the proximal shoulder 41.

With reference to FIGS. 2, 5A-5B, or 9A-9B, the plunger rod 2 is arranged within the locker 3, and more specifically within the biasing means. The plunger rod 2 includes a distal end 22 for pushing the stopper 107 inside the barrel 101 of the medical container 100, and an opposite proximal end 23 which is remote from the transversal wall 14 of the housing 10 when the plunger rod 2 is in the storage position. In the activation position, the proximal end 23 may, or not, abut against the transversal wall 14. The axial gap between the proximal end 23 of the plunger rod 2 in the storage position and the proximal end 11 of the housing 10 is equal to or higher than the axial distance that the plunger rod 2 has to travel from the storage position to activation position. The distal abutment surface 20 of the plunger rod 2 is defined by a distal side of a radial flange 21. The radial flange 21 preferably extends 360° around the plunger rod 2. The distal abutment surface 20 may be inclined with respect to the longitudinal axis A. Thus, the distal abutment surface 20 may deflect the first blocking member 31 under the action of the biasing means, and the first blocking member 31 thus can move from the blocking position to the release position as soon as the locker 3 reaches the activation position, i.e. as soon as the first blocking member 31 no longer abuts against the lateral abutment surface 40 of the retainer 4. An opposite proximal side of the radial flange 21 may serve as a support for a distal end of the biasing means.

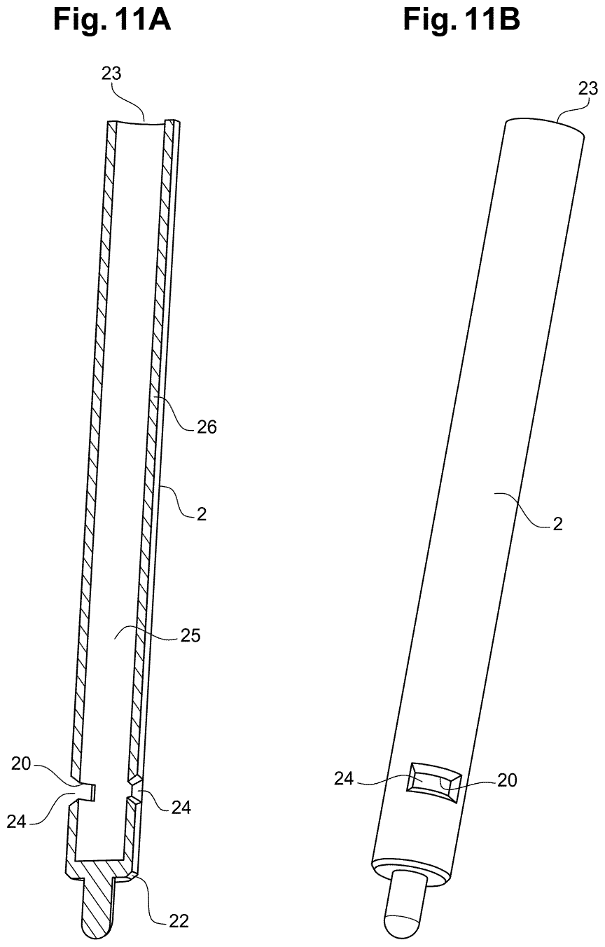

In another embodiment illustrated in FIG. 11A-11B, the biasing means may be arranged inside the plunger rod 2, instead of extending around the plunger rod 2. The plunger rod 2 is in the form of a tubular sleeve defining an inner cavity 25 for receiving the biasing means. The inner cavity 25 may define an opened proximal end 23 allowing insertion of the biasing means within the plunger rod 2 and a closed distal end 22 providing support for a distal end 61 of the biasing means. A lateral wall 26 of the plunger rod 2 may include radial openings 24 configured for receiving the first blocking members 31 of the locker 3. The distal abutment surface 20 may be formed by the top side of said openings 24.

The operation of the autoinjector 1 is described below.

Before activation of the autoinjector 1, the needle cover 5 is in the first extended position, the plunger rod 2 and the locker 3 are in the storage position, the injection spring 6 is partially compressed, the first blocking member 31 of the locker 3 is in the blocking position and cannot move to the release position because of the abutment against the lateral abutment surface 40 of the retainer 4. Besides, the injection spring 6 maintains the locker 3 in the storage position and presses the second blocking member 33 against the proximal shoulder 41. The biasing means oppose any inadvertent movement of the locker 3 in the proximal direction because of a drop test or any accidental drop. The plunger rod 2 is prevented by the locker 3 from moving in the distal direction (FIGS. 5A-5B and 9A-9B). The autoinjector 1 is prevented from being activated.

The user first has to withdraw the cap from the bottom body 10B. This withdrawal entails removal of the needle shield 104. During the cap removal, the ring may abut against the proximal shoulder 41 of the cam 7 to stop distal movement of the medical container 100.

The user then places the autoinjector 1 against the injection site. The distal end of the needle cover 5 comes against the injection site. As the user presses the autoinjector 1 against the injection site, the needle cover 5 moves from the first extended position to the retracted position. This movement of the needle cover 5 in the proximal direction causes the radial tabs 52 to slide against the cam 7. This causes the cam 7 to rotate so that the ring is shifted away from the proximal shoulder 41 and now engages the axial portion. When the needle cover 5 reaches the retracted position, the radial tab 52 is at the second end of the first slot 70.

The proximal movement of the needle cover 5 towards the retracted position also causes the proximal pushing surface 51 of the needle cover 5 to push against the (driving legs 32 of the) locker 3 and consequently to push the locker 3 together with the plunger rod 2, against the action of the injection spring 6, from the storage position to the activation position. Therefore, the first blocking member 31 moves proximally and no longer abuts against the lateral abutment surface 40 of the retainer 4 (FIGS. 6A, 10A). The flexible legs 311 are now free to deflect radially outwardly. Due to the distal force exerted by the injection spring 6 on the plunger rod 2, and due to the contact between the plunger rod 2 and the first blocking member 31, the flexible legs 311 outwardly deflect. The first blocking member 31 thus moves to the release position, i.e. is moved away from the distal abutment surface 20 of the plunger rod 2, which can move towards the injection end position under the action of the injection spring 6.

The plunger rod 2 coupled to the stopper 107 first pushes the medical container 100 in the distal direction so that the injection needle 103 reaches the injection site. This movement causes the ring to slide against the oblique portion of the second slot 71, so that the cam 7 rotates further. The radial tabs 52 reach the third end of the first slot 70; the needle cover 5 has now the ability to move distally towards the safety position. The plunger rod 2 then pushes the stopper 107 inside the barrel 101 to expel the medical product. When the stopper 107 eventually abuts against the distal end of the reservoir defined by the barrel 101, the injection is complete. An indicator, such as a visual, tactile or audible indicator, may inform the user that the injection is complete.

The user then moves the autoinjector 1 away from the injection site. The radial tabs 52 slide along the axial portion as the needle cover 5 moves from the retracted position to the safety position under the action of the safety spring 8. A locking mechanism may lock the needle cover 5 in the safety position to prevent needle stick injuries.

It is readily understandable from the above description that the autoinjector 1 of the invention permits to reduce the steps that are necessary to trigger the injection (the user has no need to push a button and the locker 3 automatically slides to the activation position when the autoinjector 1 is being pressed against the skin of the user). Due to the resistance of the biasing means, inadvertent activations are avoided and the user can get a continuous feedback (without hard points and sudden release) that the activation process is being engaged, i.e. that the autoinjector 1 is on the point to be activated.

It is to be understood that the present invention is not limited to the embodiments described above and illustrated in the drawings; rather, the skilled person will recognize that many changes and modifications may be made within the scope of the appended claims.

1. An autoinjector for automatic injection of a product into an injection site, said autoinjector comprising:
a housing extending along a longitudinal axis A and configured to receive a medical container having a barrel defining a reservoir for containing a medical product, said barrel having a distal end provided with an injection needle and an opened proximal end configured to receive a plunger rod for pushing a stopper arranged inside the barrel,
a needle cover coupled to and axially movable with respect to said housing between a first extended position, in which the needle cover at least partially shields the injection needle, a retracted position, in which the needle cover moves proximally with respect to the housing, and a second extended position in which the needle cover moves back in the distal direction to shield the injection needle,
plunger rod axially movable inside the housing between a storage position, an activation position proximally located relative to the storage position, and an injection end position distally located relative to the storage position and the activation position, the plunger rod being configured to push the stopper in order to expel the medical product when moving from the activation position to the injection end position,
a biasing member biasing the plunger rod in a distal direction towards the injection end position,
a locker for distally blocking the plunger rod in the storage position before activation of the autoinjector, wherein the locker includes a first blocking member radially movable between a blocking position, in which said first blocking member axially abuts against a distal abutment surface of the plunger rod for preventing distal movement of the plunger rod with respect to the locker, and a release position, in which the first blocking member is away from the distal abutment surface of the plunger rod to allow for distal movement of the plunger rod with respect to the locker,
a retainer secured to the housing for maintaining the first blocking member of the locker in the blocking position, the retainer comprising a lateral abutment surface for radially abutting against the locker such that the first blocking member of the locker is blocked in the blocking position, and the retainer comprising a proximal shoulder for axially abutting against a second blocking member of the locker,
wherein the locker is axially movable relative to the retainer together with the plunger rod between a storage position, in which the locker radially abuts against the lateral abutment surface of the retainer, such that the first blocking member is blocked in the blocking position, and in which the second blocking member of the locker axially abuts against the proximal shoulder of the retainer such that the retainer distally blocks the locker in said storage position, and an activation position, proximally located with respect to the storage position, in which the locker is away from the lateral abutment surface of the retainer, such that the first blocking member of the locker can move from the blocking position to the release position, movement of the locker from the storage position to the activation position being caused by the needle cover moving from the first extended position to the retracted position.

2. The autoinjector according to claim 1, wherein the first blocking member defines a proximal abutment surface for axially abutting against the distal abutment surface of the plunger rod, said proximal abutment surface of the first blocking member and/or said distal abutment surface of the plunger rod being inclined.

3. The autoinjector according to claim 1, wherein the first blocking member is arranged on an inner side of an axially extending flexible leg of the locker, and an outer side of the flexible leg defines an outer abutment surface configured for abutting against the lateral abutment surface of the retainer when the locker is in the storage position.

4. The autoinjector according to claim 3, wherein the first blocking member is located at a distal end of the flexible leg, and the flexible leg is delimited between two axial slots.

5. The autoinjector according to claim 3, wherein the second blocking member is located on an outer surface of the flexible leg of the locker.

6. The autoinjector according to claim 1, wherein the locker comprises a driving leg for receiving a proximal push of the needle cover when the needle cover moves from the first extended position to the retracted position.

7. The autoinjector according to claim 1, wherein the locker has a cylindrical guiding portion and the retainer has one or more cylindrical centering surfaces configured for guiding axial movement of the locker inside the retainer between the storage position and the activation position.

8. The autoinjector according to claim 2, wherein the guiding portion of the locker comprises anti-rotation means for blocking rotation of the locker relative to the retainer.

9. The autoinjector according to claim 7, wherein the proximal shoulder is arranged proximal to the one or more centering surfaces.

10. The autoinjector according to claim 1, wherein the lateral abutment surface of the retainer is defined by a radially inward protrusion, and proximal to said radially inward protrusion, the retainer has a portion of larger diameter configured for receiving the first blocking member when the first blocking member of the locker moves to the release position.

11. The autoinjector according to claim 10, wherein the proximal shoulder of the retainer is located on a proximal side of the radially inward protrusion of the retainer.

12. The autoinjector according to claim 1, wherein the second blocking member is a resiliently deformable tab.

13. The autoinjector according to im 1, wherein the housing has a containment wall configured to laterally retain a proximal end of the biasing member.

14. The autoinjector according to claim 1, wherein the retainer and the housing are made of a single piece.

15. The autoinjector according to claim 1, wherein movement of the locker from the storage position to the activation position is opposed by the biasing as member only.
